(19) United States
(12) Patent Application Publication
(10) Pub. No.: US 2020/0315496 A1
(43) Pub. Date: Oct. 8, 2020

(54) INFORMATION PROCESSING APPARATUS, INFORMATION PROCESSING METHOD, AND INFORMATION PROCESSING PROGRAM

(71) Applicant: OMRON HEALTHCARE CO., LTD., Muko-shi (JP)

(72) Inventors: Toru DENO, Kyoto (JP); Hiroshi USUI, Kyoto (JP); Kosuke INOUE, Kyoto (JP); Yasushi MATSUOKA, Kyoto (JP); Yoshiyuki MORITA, Kyoto-shi (JP); Naoki TSUCHIYA, Kyoto-shi (JP)

(73) Assignee: OMRON HEALTHCARE CO., LTD., Muko-shi (JP)

(21) Appl. No.: 16/910,213

(22) Filed: Jun. 24, 2020

Related U.S. Application Data
(63) Continuation of application No. PCT/JP2018/046241, filed on Dec. 17, 2018.

(30) Foreign Application Priority Data
Dec. 27, 2017 (JP) 2017-252479

(51) Int. Cl. A61B 5/11; A61B 5/021; A61B 5/00

(52) U.S. Cl. CPC A61B 5/1118; A61B 5/681; A61B 5/021; A61B 5/112

(57) ABSTRACT

According to one embodiment, an information processing apparatus includes a signal acquisition unit that acquires a signal representing a movement of a subject from a sensor that detects the movement of the subject, a measurement unit that measures at least one of an activity amount and a number of steps of the subject based on the signal representing the movement of the subject, and an estimation unit that estimates a situation of the subject based on at least one of the activity amount or the number of steps.

### CROSS-REFERENCE TO RELATED APPLICATIONS

This application is a Continuation Application of PCT Application No. PCT/JP2018/046241, filed Dec. 17, 2018 and based upon and claiming the benefit of priority from Japanese Patent Application No. 2017-252479, filed Dec. 27, 2017, the entire contents of all of which are incorporated herein by reference.

### BACKGROUND OF THE INVENTION

### Field

The present invention relates generally to an information processing apparatus, an information processing method, and an information processing program for estimating a situation of a subject.

### Background

In recent years, a wearable blood pressure monitor capable of measuring blood pressure regardless of place has been developed. Jpn. Pat. Appln. KOKAI Publication No. 2017-023546 discloses a wearable blood pressure monitor that starts blood pressure measurement in response to an input of a blood pressure measurement start instruction.

There is also interest in events that cause high blood pressure in specific situations. One of such situations is the phenomenon known as workplace hypertension, in which the blood pressure value is normal at home but becomes high in a workplace. It is therefore desirable for the subject of blood pressure measurement to periodically measure his or her own blood pressure while staying at work in order to confirm the existence of work hypertension.

### SUMMARY

In a first aspect of the present invention, there is provided an information processing apparatus including: a signal acquisition unit configured to acquire a signal representing a movement of a subject from a sensor configured to detect the movement of the subject; a measurement unit configured to measure at least one of an activity amount or a number of steps of the subject based on the signal representing the movement of the subject; and an estimation unit configured to estimate a situation of the subject based on at least one of the activity amount or the number of steps.

According to the first aspect of the present invention, since the information processing apparatus can estimate the situation of a measurement subject by referring to information from an already mounted sensor, the situation of the measurement subject can be estimated with a simple configuration. In addition, since the information processing apparatus does not need to refer to an external signal such as a global positioning system (GPS) signal, even in a case where the GPS signal cannot be acquired, the situation of the measurement subject can be estimated. In addition, the information processing apparatus does not need to register position information of various places for estimating the situation of the measurement subject in a memory as in the case of estimating the situation of the measurement subject based on the GPS signal. Therefore, the information processing apparatus can effectively utilize memory resources. Furthermore, for example, the information processing apparatus can acquire a blood pressure value in an estimated situation. As a result, the measurement subject can confirm their suspicion of high blood pressure in the estimated situation at an early stage.

According to a second aspect of the present invention, as the situation of the subject, the estimation unit estimates that the subject is moving/staying based on a variation in at least one of the activity amount per unit time or the number of steps per unit time in the information processing apparatus according to the first aspect.

According to the second aspect of the present invention, the information processing apparatus can provide estimation results of different situations. Furthermore, for example, the information processing apparatus can acquire a blood pressure value while the subject is moving and a blood pressure value while the subject stays. As a result, the subject can confirm their suspicion of high blood pressure while moving (for example, while riding a train) at an early stage. Similarly, the subject may be able to confirm their suspicion of high blood pressure during a stay at a place at an early stage.

According to a third aspect of the present invention, in the information processing apparatus according to the first or the second aspect, the information processing apparatus further includes a setting acquisition unit configured to acquire life pattern data including a scheduled staying time zone related to at least one place of the subject, and in a case where the estimation unit estimates that the subject is staying, the estimation unit estimates a staying place of the subject with reference to the life pattern data.

According to the third aspect of the present invention, the information processing apparatus can accurately estimate the staying place of the subject. For example, the information processing apparatus can acquire a blood pressure value at each staying place of the subject. As a result, the subject can confirm their suspicion of high blood pressure at each staying place (for example, a workplace where high blood pressure is likely to occur) at an early stage.

According to a fourth aspect of the present invention, an information processing apparatus further includes: a designation information acquisition unit configured to acquire designation information including a designated place based on designation by the subject and a past staying date and time range at the designated place; and a creation unit configured to create an estimation condition used for estimating that the subject is staying at the designated place based on at least one of the activity amount or the number of steps in a time zone including the staying date and time range.

According to the fourth aspect of the present invention, the information processing apparatus can accurately estimate that the subject is staying at the designated place by referring to the estimation condition based on at least one of the activity amount or the number of steps actually measured.

A fifth aspect of the present invention is an information processing method including: acquiring a signal representing a movement of a subject from a sensor that detects the movement of the subject; measuring at least one of an activity amount or a number of steps of the subject based on the signal representing the movement of the subject; and estimating a situation of the subject based on at least one of the activity amount or the number of steps.

According to the fifth aspect of the present invention, the information processing method can obtain the same effect as the first aspect described above. That is, the information processing method can estimate the situation of the subject.

A sixth aspect of the present invention is an information processing program for causing a computer to function as each unit included in the information processing apparatus according to any one of the first to fourth aspects.

According to the sixth aspect of the present invention, the information processing program can obtain the same effect as the first aspect described above. That is, the information processing program can estimate the situation of the subject.

According to the present invention, it is possible to provide a technique that is capable of estimating a situation of a subject.

### BRIEF DESCRIPTION OF THE DRAWINGS

FIG. 1 shows an appearance of a blood pressure monitor according to an embodiment.

FIG. 2 is a block diagram of a blood pressure monitor according to an embodiment.

FIG. 3 is a cross-sectional view of a blood pressure monitor according to an embodiment.

FIG. 4 is a functional block diagram of a blood pressure monitor according to an embodiment.

FIG. 5 shows an example of a plurality of life pattern candidates according to an embodiment.

FIG. 6 is a flowchart showing a procedure for estimating a situation of a measurement subject according to an embodiment.

FIG. 7 is a distribution diagram of an activity amount measured by a blood pressure monitor according to an embodiment.

### DETAILED DESCRIPTION

Hereinafter, embodiments according to the present invention will be described with reference to the drawings.

The situation in which a blood pressure value of a subject is acquired has to be determined and managed by the subject himself/herself. Therefore, a technique to estimate the situation of the subject is desired.

An object to be accomplished by the embodiments is to provide an information processing apparatus, an information processing method, and an information processing program for estimating the situation of the subject.

### First Embodiment

(Configuration of Blood Pressure Monitor)

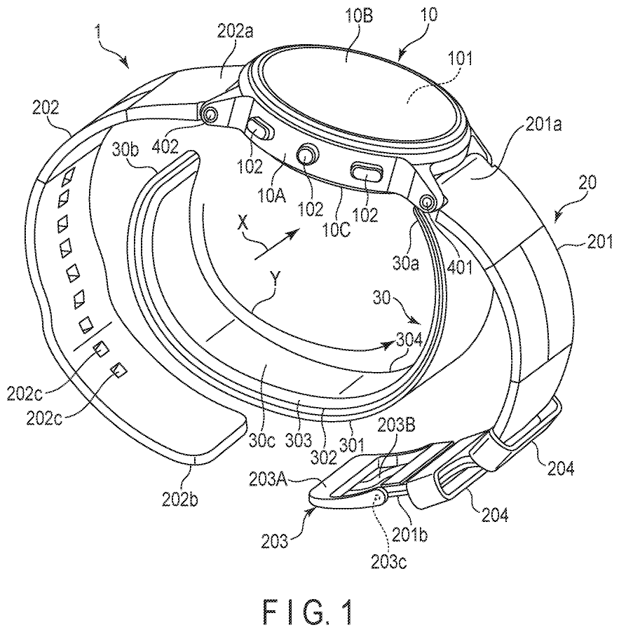

FIG. 1 shows an appearance of a blood pressure monitor 1 as an embodiment of an information processing apparatus according to the present invention. The blood pressure monitor 1 is a wristwatch-type wearable device. The blood pressure monitor 1 has a blood pressure measurement function as a blood pressure measurement unit, and further has various information processing functions. The information processing function includes, for example, an activity amount measurement function, a step count measurement function, a sleep state measurement function, and an environment (temperature and humidity) measurement function. The blood pressure monitor 1 is, for example, a blood pressure monitor of a type that starts blood pressure measurement based on an input of a blood pressure measurement start instruction by a measurement subject or a trigger signal autonomously generated by the blood pressure monitor 1. The measurement subject is an example of a subject to be a target of situation estimation performed by the blood pressure monitor 1 described below.

The blood pressure monitor 1 includes a main body 10, a belt 20, and a cuff structure 30.

The configuration of the main body 10 will be described.

The main body 10 is configured to be able to mount a plurality of elements such as elements of a control system of the blood pressure monitor 1.

The main body 10 includes a case 10A, a glass 10B, and a back cover 10C.

The case 10A has, for example, a substantially short cylindrical shape. The case 10A is provided with a pair of projecting lugs for attaching the belt 20 at two positions on its side surface.

The glass 10B is attached to an upper portion of the case 10A. The glass 10B has, for example, a circular shape.

The back cover 10C is detachably attached to the lower portion of the case 10A so as to face the glass 10B.

On the main body 10 is mounted a display unit 101 and an operation unit 102.

The display unit 101 displays various kinds of information. The display unit 101 is provided in the main body 10 at a position that can be visually recognized by the measurement subject through the glass 10B. The display unit 101 is, for example, a liquid crystal display (LCD). The display unit 101 may be an organic electro luminescence (EL) display. The display unit 101 may have a function of displaying various kinds of information, and is not limited thereto. The display unit 101 may include a light emitting diode (LED).

The operation unit 102 is an element for inputting various instructions to the blood pressure monitor 1. The operation unit 102 is provided on a side surface of the main body 10. The operation unit 102 includes, for example, one or more push switches. The operation unit 102 may be a pressure-sensitive (resistive) or proximity (capacitive) touch panel switch. The operation unit 102 may have a function of inputting various instructions to the blood pressure monitor 1, and is not limited thereto.

Examples of switches included in the operation unit 102 will be described.

The operation unit 102 includes a measurement switch for instructing start or stop of blood pressure measurement. The operation unit 102 may include a home switch for returning the display screen of the display unit 101 to a predetermined home screen, and a record call switch for causing the display unit 101 to display a measurement record of the past blood pressure, activity amount, and the like.

The main body 10 includes a plurality of elements other than the display unit 101 and the operation unit 102. A plurality of elements mounted on the main body 10 will be described later.

The configuration of the belt 20 will be described.

The belt 20 is configured to be able to surround a measurement site (for example, a left wrist) of the measurement subject. The width direction of the belt 20 is defined as an X direction. The direction in which the belt 20 surrounds the measurement site is a Y direction.

The belt 20 includes a first belt portion 201, a second belt portion 202, a buckle 203, and a belt holding portion 204.

The first belt portion 201 has a band shape extending from the main body 10 to one side in one direction (right side in FIG. 1). A root portion 201a of the first belt portion 201 close to the main body 10 is rotatably attached to a pair of lugs of the main body 10 via a connecting rod 401.

The second belt portion 202 has a band shape extending from the main body 10 to the other side in one direction (left side in FIG. 1). A root portion 202a of the second belt portion 202 close to the main body 10 is rotatably attached to a pair of lugs of the main body 10 via a connecting rod 402. A plurality of small holes 202c are formed between the root portion 202a and a distal end portion 202b of the second belt portion 202 remote from the main body 10 so as to penetrate the second belt portion 202 in the thickness direction.

The buckle 203 is configured to be able to fasten the first belt portion 201 and the second belt portion 202. The buckle 203 is attached to a distal end portion 201b of the first belt portion 201 that is far from the main body 10. The buckle 203 includes a frame-shaped body 203A, a fastening rod 203B, and a connecting rod 203C.

The frame-shaped body 203A and the fastening rod 203B are rotatably attached to the distal end portion 201b of the first belt portion 201 via the connecting rod 203C. The frame-shaped body 203A and the fastening rod 203B are made of, for example, a metal material. The frame-shaped body 203A and the fastening rod 203B may be made of a plastic material. When the first belt portion 201 and the second belt portion 202 are fastened, the distal end portion 202b of the second belt portion 202 is passed through the frame-shaped body 203A. The fastening rod 203B is inserted into any one of the plurality of small holes 202c of the second belt portion 202.

The belt holding portion 204 is attached between the root portion 201a and the distal end portion 201b of the first belt portion 201. When the first belt portion 201 and the second belt portion 202 are fastened, the distal end portion 202b of the second belt portion 202 is passed through the belt holding portion 204.

The configuration of the cuff structure 30 will be described.

The cuff structure 30 is configured to be able to press a measurement site during blood pressure measurement.

The cuff structure 30 has a band shape extending along the Y direction. The cuff structure 30 faces the inner peripheral surface of the belt 20. One end 30a of the cuff structure 30 is attached to the main body 10. The other end 30b of the cuff structure 30 is a free end. Therefore, the cuff structure 30 can be separated from the inner peripheral surface of the belt 20.

The cuff structure 30 includes a curler 301, a pressing cuff 302, a back plate 303, and a sensing cuff 304.

The curler 301 is disposed at the outermost periphery of cuff structure 30. The curler 301 is curved along the Y direction in a natural state. The curler 301 is a resin plate having predetermined flexibility and hardness. The resin plate is made of, for example, polypropylene.

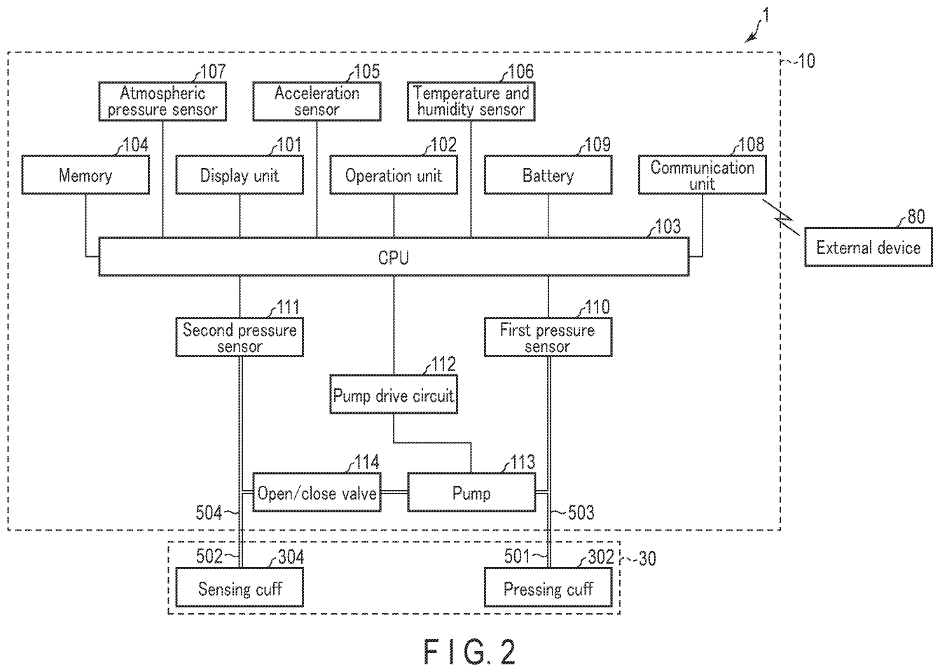

The pressing cuff 302 is disposed along the inner peripheral surface of the curler 301. The pressing cuff 302 has a bag shape. A flexible tube 501 (shown in FIG. 2) is attached to the pressing cuff 302. The flexible tube 501 is an element for supplying a pressure transmission fluid (hereinafter also simply referred to as “fluid”) from the main body 10 side or discharging the fluid from the pressing cuff 302. The fluid is, for example, air. When the fluid is supplied to the pressing cuff 302, the pressing cuff 302 is inflated and presses the measurement site.

The pressing cuff 302 may include, for example, two fluid bags stacked in the thickness direction. Each fluid bag is formed of, for example, a stretchable polyurethane sheet. As fluid is supplied to the pressing cuff 302, the fluid flows into each fluid bag. As each fluid bag inflates, the pressing cuff 302 inflates.

The back plate 303 is disposed along the inner peripheral surface of the pressing cuff 302. The back plate 303 has a band shape. The back plate 303 is made of, for example, resin. The resin is, for example, polypropylene. The back plate 303 functions as a reinforcing plate. Therefore, the back plate 303 can transmit the pressing force from the pressing cuff 302 to the entire region of the sensing cuff 304.

On the inner peripheral surface and the outer peripheral surface of the back plate 303, a plurality of grooves having a V-shaped or U-shaped cross section extending in the direction X are provided in parallel to be spaced apart from each other in the direction Y. Because the back plate 303 is flexible, the back plate 303 does not prevent the cuff structure 30 from attempting to bend.

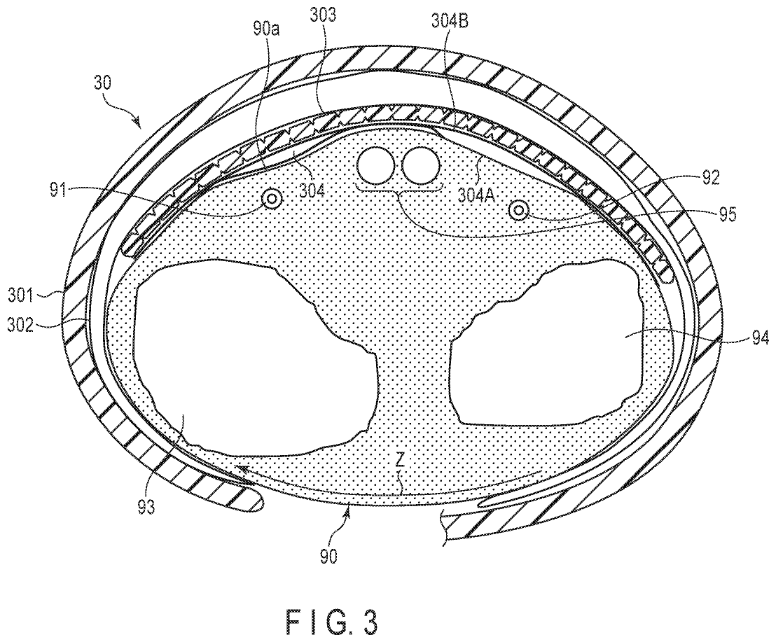

The sensing cuff 304 is disposed along the inner peripheral surface of the back plate 303. The sensing cuff 304 has a bag shape. The sensing cuff 304 includes a first sheet 304A (shown in FIG. 3) and a second sheet 304B (shown in FIG. 3) facing the first sheet 304A. The first sheet 304A corresponds to the inner peripheral surface 30c of the cuff structure 30. Therefore, the first sheet 304A contacts the measurement site. The second sheet 304B faces the inner peripheral surface of the back plate 303. The first sheet 304A and the second sheet 304B are, for example, stretchable polyurethane sheets. A flexible tube 502 (shown in FIG. 2) is attached to sensing cuff 304. The flexible tube 502 is an element for supplying fluid to the sensing cuff 304 or discharging fluid from the sensing cuff 304.

A plurality of elements mounted on the main body 10 will be described.

FIG. 2 is a block diagram of the blood pressure monitor 1.

The main body 10 includes a central processing unit (CPU) 103, a memory 104, an acceleration sensor 105, a temperature and humidity sensor 106, an atmospheric pressure sensor 107, a communication unit 108, a battery 109, a first pressure sensor 110, a second pressure sensor 111, a pump drive circuit 112, a pump 113, and an open/close valve 114 in addition to the display unit 101 and the operation unit 102.

The CPU 103 is an example of a processor constituting a computer. The CPU 103 executes various functions as a controller according to a program stored in the memory 104, and controls the operation of each unit of the blood pressure monitor 1. The configuration of each unit mounted on the CPU 103 will be described later.

The memory 104 stores a program that causes the CPU 103 to function as each unit included in the blood pressure monitor 1. The program may be referred to as a command to operate the CPU 103. Furthermore, the memory 104 stores data used for controlling the blood pressure monitor 1, setting data for setting various functions of the blood pressure monitor 1, data of measurement results of blood pressure values, and the like. The memory 104 is used as a work memory, etc. when the program is executed.

The acceleration sensor 105 is a three-axis acceleration sensor. The acceleration sensor 105 outputs acceleration signals representing accelerations in three directions orthogonal to each other to the CPU 103. Using the acceleration signal, the CPU 103 can calculate the amount of activity of the measurement subject not only in walking but also in various activities such as housework and desk work. The activity amount is, for example, an index related to the activity of the measurement subject such as a moving (walking) distance, calorie consumption, or a fat burning amount. The CPU 103 can also estimate the sleep state by detecting the state in which the measurement subject turns over in bed using the acceleration signal.

The temperature and humidity sensor 106 measures the environmental temperature and humidity around the blood pressure monitor 1. The temperature and humidity sensor 106 outputs environmental data representing the environmental temperature and humidity to the CPU 103. The CPU 103 stores the environmental data in the memory 104 in association with the measurement time of the temperature and humidity sensor 106. For example, temperature (change in temperature) is considered as a factor that can cause blood pressure fluctuation in a human. Therefore, the environmental data is information that can be a factor of blood pressure fluctuation of the measurement subject. The atmospheric pressure sensor 107 detects atmospheric pressure.

The atmospheric pressure sensor 107 outputs atmospheric pressure data to the CPU 103. The CPU 103 can measure the number of steps, the number of fast walking steps, the number of stair climbing steps, and the like of the measurement subject using the atmospheric pressure data and the acceleration signal.

The communication unit 108 is an interface for connecting the blood pressure monitor 1 to an external device 80. The external device 80 is, for example, a mobile terminal such as a smartphone or a tablet terminal, or a server. The communication unit 108 is controlled by the CPU 103. The communication unit 108 transmits information to the external apparatus 80 via the network. The communication unit 108 transfers information received from the external apparatus 80 via the network to the CPU 103. The communication via the network may be either wireless or wired. The network is, for example, the Internet, but is not limited thereto. The network may be another type of network such as a hospital local area network (LAN), or may be a one-to-one communication using a USB cable or the like. The communication unit 108 may include a micro USB connector. The communication unit 108 may transmit information to the external apparatus 80 by short-range wireless communication such as Bluetooth (registered trademark).

The battery 109 is, for example, a rechargeable secondary battery. The battery 109 supplies power to each element mounted on the main body 10. The battery 109 supplies power to, for example, the display unit 101, the operation unit 102, the CPU 103, the memory 104, the acceleration sensor 105, the temperature and humidity sensor 106, the atmospheric pressure sensor 107, the communication unit 108, the first pressure sensor 110, the second pressure sensor 111, the pump drive circuit 112, the pump 113, and the open/close valve 114.

The first pressure sensor 110 is, for example, a piezoresistive pressure sensor. The first pressure sensor 110 detects the pressure in the pressing cuff 302 via the flexible tube 501 and a first flow path forming member 503 constituting a first flow path. The first pressure sensor 110 outputs pressure data to the CPU 103.

The second pressure sensor 111 is, for example, a piezoresistive pressure sensor. The second pressure sensor 111 detects the pressure in the sensing cuff 304 via the flexible tube 502 and a second flow path forming member 504 constituting a second flow path. The second pressure sensor 111 outputs pressure data to the CPU 103.

The pump drive circuit 112 drives the pump 113 based on a control signal from the CPU 103.

The pump 113 is, for example, a piezoelectric pump. The pump 113 is fluidly connected to the pressing cuff 302 via the first flow path. The pump 113 can supply fluid to the pressing cuff 302 through the first flow path. The pump 113 is equipped with an exhaust valve (not shown) which is controlled to open and close in accordance with ON/OFF of the pump 113. That is, the exhaust valve closes when the pump 113 is turned on to assist in enclosing the fluid within the pressing cuff 302. On the other hand, when the pump 113 is turned off, the exhaust valve is opened to discharge the fluid in the pressing cuff 302 to the atmosphere through the first flow path. The exhaust valve has a function of a check valve, and the discharged fluid does not flow back.

The pump 113 is further fluidly connected to the sensing cuff 304 via the second flow path. The pump 113 may supply fluid to the sensing cuff 304 through the second flow path.

The open/close valve 114 is interposed in the second flow path forming member 504. The open/close valve 114 is, for example, a normally-open electromagnetic valve. Opening and closing (opening degree) of the open/close valve 114 is controlled based on a control signal from the CPU 103. When the open/close valve 114 is in an open state, the pump 113 can supply fluid to the sensing cuff 304 through the second flow path.

A state in which the blood pressure monitor 1 is attached to the measurement site (hereinafter also referred to as “attached state”) will be described.

FIG. 3 is a view showing a cross section perpendicular to the left wrist 90 which is the measurement site in the attached state. The main body 10 and the belt 20 are not shown. FIG. 3 shows the radial artery 91, ulnar artery 92, radius 93, ulna 94, and tendon 95 of the left wrist 90.

In this worn state, curler 301 extends along the outer periphery (Z direction) of left wrist 90. The pressing cuff 302 extends along the Z direction on the inner peripheral side of the curler 301. The back plate 303 is interposed between the pressing cuff 302 and the sensing cuff 304, and extends along the Z direction. The sensing cuff 304 is in contact with the left wrist 90 and extends in the Z direction so as to cross the artery passing portion 90a of the left wrist 90. The belt 20, the curler 301, the pressing cuff 302, and the back plate 303 function as a pressing member capable of generating a pressing force toward the left wrist 90, and press the left wrist 90 via the sensing cuff 304.

The configuration of each unit implemented by the CPU 103 will be described.

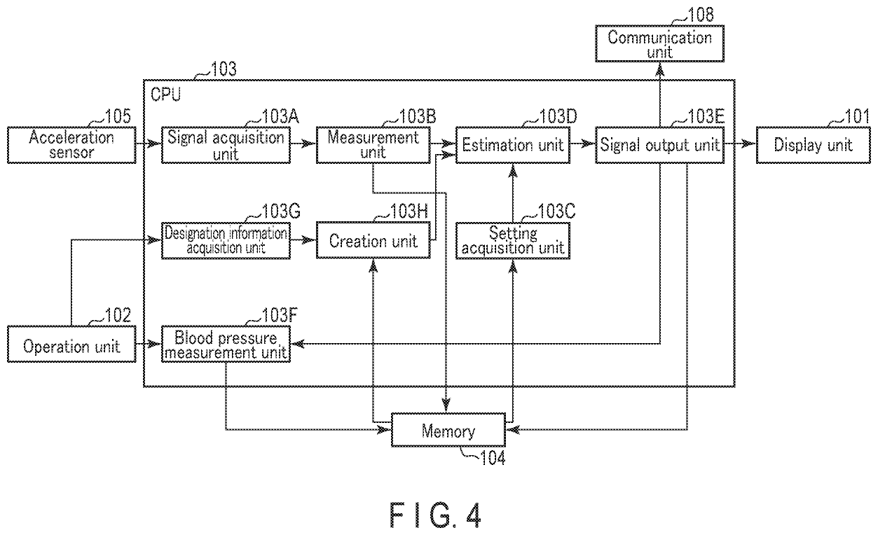

FIG. 4 is a functional block diagram of the blood pressure monitor 1. The CPU 103 implements a signal acquisition unit 103A, a measurement unit 103B, a setting acquisition unit 103C, an estimation unit 103D, a signal output unit 103E, a blood pressure measurement unit 103F, a designation information acquisition unit 103G, and a creation unit 103H. Note that each unit may be distributed and implemented in two or more processors.

The configuration of the signal acquisition unit 103A will be described.

The signal acquisition unit 103A acquires an acceleration signal from the acceleration sensor 105. The acceleration sensor 105 is an example of a sensor that detects the movement of the measurement subject. The acceleration signal is an example of a signal representing the movement of the measurement subject. The signal acquisition unit 103A sequentially outputs the acceleration signals sequentially acquired from the acceleration sensor 105 to the measurement unit 103B.

The configuration of the measurement unit 103B will be described.

The measurement unit 103B measures (calculates) at least one of the activity amount or the number of steps of the measurement subject based on the acceleration signal. The measurement unit 103B outputs at least one of activity amount data or step count data to the estimation unit 103D. For example, the measurement unit 103B can output the activity amount data per unit time to the estimation unit 103D each time the activity amount per unit time is measured. Similarly, every time the measuring unit 103B measures the number of steps per unit time, the measuring unit 103B can output the step count data per unit time to the estimating unit 103D. The length of the unit time can be arbitrarily set.

The measurement unit 103B stores the activity amount data per unit time and the step count data per unit time in the memory 104 in association with the measurement time.

The configuration of the setting acquisition unit 103C will be described.

The setting acquisition unit 103C acquires life pattern data of the measurement subject set in advance by the measurement subject from the memory 104. The setting acquisition unit 103C outputs the life pattern data to the estimation unit 103D. The life pattern data is registered in the memory 104 based on the setting of a life pattern by the measurement subject using the operation unit 102.

Here, the life pattern data will be described.

The life pattern data is a standard of the behavior of the measurement subject. The life pattern data is used for estimation of the situation of the measurement subject by the estimation unit 103D described later. The situation of the measurement subject is, for example, “moving” and “staying”, but is not limited thereto.

The life pattern data includes a scheduled staying time zone related to at least one place of the measurement subject. For example, the life pattern data may include at least a scheduled staying time zone in a workplace or school to which the measurement subject commutes. Note that the term “workplace” in the following description may be read as “workplace or school” as appropriate. For example, the life pattern data may include at least a scheduled staying time zone at home. The life pattern data may include a scheduled staying time zone in at least one place other than the home and the workplace.

The scheduled staying time zone is a unit such as daytime or nighttime. Here, as an example, it is assumed that the daytime is a predetermined time zone extending over 12 p.m. and the nighttime is a predetermined time zone extending over 0 a.m. The scheduled staying time zone may be a specific time zone in which a start time and an end time are designated instead of a unit such as daytime or nighttime. In a case where the life pattern data includes scheduled staying time zones at two or more places, the scheduled staying time zones at the two or more places are time zones that do not overlap each other. This is because the estimation unit 103D estimates the staying place of the measurement subject with reference to the life pattern data. If there are one or more overlapping time zones for the scheduled staying time zones of the two or more places, the estimation unit 103D cannot estimate the staying place of the measurement subject.

The lifestyle pattern data may include days of the week of attendance related to workplace or school. Note that the term “attendance” in the following description may be read as “attendance at work or school” as appropriate. The life pattern data may include a day of the week of staying at a place different from the workplace.

The life pattern data may include matters other than those described above. For example, the life pattern data is set for a single model case of an arbitrary day of the measurement subject. The life pattern data may be set for each day of the week instead of for a single model case.

For example, the life pattern data is set by the measurement subject selecting a life pattern candidate close to his/her own life pattern from among a plurality of life pattern candidates. Some examples of the life pattern candidates will be described later. The life pattern data may be set by the measurement subject inputting each item of the life pattern data instead of the measurement subject selecting the life pattern candidate.

The configuration of the estimation unit 103D will be described.

The estimation unit 103D estimates the situation of the measurement subject based on at least one of the activity amount or the number of steps of the measurement subject measured by the measurement unit 103B. The estimation of the situation of the measurement subject performed by the estimation unit 103D based on at least one of the activity amount or the number of steps will be described later. Furthermore, in a case where the estimation unit 103D estimates the situation of the measurement subject as staying, the estimation unit 103D estimates the staying place of the measurement subject by referring to the life pattern data. The estimation unit 103D can also estimate the staying place of the measurement subject without referring to the life pattern data. The estimation of the staying place of the measurement subject performed by the estimation unit 103D will be described later.

The estimation unit 103D can also estimate the situation of the measurement subject based on at least one of the activity amount or the number of steps of the measurement subject with reference to the estimation condition created by the creation unit 103H described later. The estimation of the situation of the measurement subject performed by the estimation unit 103D with reference to the estimation condition will be described later.

The estimation unit 103D outputs the estimation result including the situation of the measurement subject to the signal output unit 103E. For example, the situation of the measurement subject included in the estimation result is associated with the date and time. The estimation unit 103D can acquire date and time information by a clock function of the blood pressure monitor 1.

In one example, the estimation unit 103D outputs the estimation result to the signal output unit 103E at a predetermined time interval. The predetermined time is, for example, a fixed time, but may be an arbitrarily changeable time.

In another example, the estimation unit 103D outputs the estimation result to the signal output unit 103E in the case of estimating that the situation of the measurement subject has transitioned from a first situation to a second situation. For example, in a case where the estimation unit 103D estimates that the situation of the measurement subject has transitioned from moving to staying, the estimation unit 103D outputs an estimation result including information indicating that the measurement subject is staying to the signal output unit 103E. For example, in a case where the estimation unit 103D estimates that the situation of the measurement subject has transitioned from staying to moving, the estimation unit 103D outputs an estimation result including information indicating that the measurement subject is moving to the signal output unit 103E. According to this example, since the estimation unit 103D outputs the estimation result to the signal output unit 103E less frequently, the processing load of the CPU 103 is also reduced.

The configuration of the signal output unit 103E will be described.

The signal output unit 103E receives the estimation result from the estimation unit 103D and outputs a signal based on the estimation result. Some examples of the signal based on the estimation result will be described.

In one example, the signal output unit 103E outputs, as the signal based on the estimation result, an instruction signal for instructing the measurement subject to perform support for blood pressure measurement.

The instruction signal includes an instruction for prompting the measurement subject to input an instruction to start blood pressure measurement as support for the blood pressure measurement. The signal output unit 103E outputs the instruction signal to the display unit 101. The display unit 101 displays an image prompting the measurement subject to input a blood pressure measurement start instruction based on the instruction signal. The content of the image is not limited as long as the measurement subject can recognize that an instruction to start blood pressure measurement needs to be input. As a result, the measurement subject can recognize that the blood pressure measurement needs to be performed and presses the measurement switch to start the blood pressure measurement. The blood pressure monitor 1 may prompt the measurement subject to input the instruction to start blood pressure measurement by vibration, sound, or the like based on the instruction signal.

The instruction signal may include a blood pressure measurement start instruction that triggers the blood pressure measurement unit 103F to start blood pressure measurement, instead of an instruction to prompt the measurement subject to input the blood pressure measurement start instruction. The signal output unit 103E outputs the instruction signal to the blood pressure measurement unit 103F. As a result, the blood pressure monitor 1 can start the blood pressure measurement for the measurement subject without requiring the measurement subject to input the instruction to start the blood pressure measurement. That is, for the measurement subject, the blood pressure measurement is automatically performed without performing the input operation of the measurement start instruction.

In another example, the signal output unit 103E outputs a signal including the estimation result to the memory 104 as a signal based on the estimation result. The memory 104 stores the estimation result. Accordingly, the blood pressure monitor 1 can accumulate the situation of the measurement subject in association with the date and time.

In yet another example, the signal output unit 103E outputs a signal including the estimation result to the external device 80 via the communication unit 108 as a signal based on the estimation result. The external device 80 stores the estimation result. Accordingly, the external device 80 can accumulate the situation of the measurement subject in association with the date and time.

The signal output unit 103E outputs at least one of the instruction signal or the signal including the estimation result. In the case of outputting the signal including the estimation result, the signal output unit 103E outputs the signal to at least one of the memory 104 or the external device 80.

The configuration of the blood pressure measurement unit 103F will be described.

The blood pressure measurement unit 103F controls the blood pressure measurement of the measurement subject, for example, in the manner below.

The blood pressure measurement unit 103F initializes the processing memory area of the memory 104 based on the detection of the depression of the measurement switch by the measurement subject or the detection of the instruction signal that triggers the start of the blood pressure measurement. The blood pressure measurement unit 103F turns off the pump 113 via the pump drive circuit 112, opens the exhaust valve built in the pump 113, maintains the open/close valve 114 in the open state, and performs control to exhaust the fluid in the pressing cuff 302 and the sensing cuff 304. The blood pressure measurement unit 103F controls the first pressure sensor 110 and the second pressure sensor 111 to adjust the pressure to 0 mmHg.

The blood pressure measurement unit 103F turns on the pump 113 via the pump drive circuit 112, maintains the open/close valve 114 in the open state, and performs control to start pressurization of the pressing cuff 302 and the sensing cuff 304. The blood pressure measurement unit 103F controls the pump 113 to be driven via the pump drive circuit 112 while monitoring the pressures of the pressing cuff 302 and the sensing cuff 304 by the first pressure sensor 110 and the second pressure sensor 111, respectively. The blood pressure measurement unit 103F controls the fluid to be sent to the pressing cuff 302 through the first flow path and to the sensing cuff 304 through the second flow path, respectively. The blood pressure measurement unit 103F waits until the pressure of the sensing cuff 304 reaches a predetermined pressure (for example, 15 mmHg), or a predetermined time (for example, three seconds) elapses for the driving time of the pump 113. The blood pressure measurement unit 103F closes the open/close valve 114 and continues the control of supplying the fluid from the pump 113 to the pressing cuff 302 through the first flow path. As a result, the pressing cuff 302 is gradually pressurized to gradually press the left wrist 90. The back plate 303 transmits the pressing force from the pressing cuff 302 to the sensing cuff 304.

The sensing cuff 304 presses the left wrist 90 (including the artery passing portion 90a). In this pressurization process, in order to calculate blood pressure values (systolic blood pressure SBP and diastolic blood pressure DBP), the blood pressure measurement unit 103F monitors pressure Pc of the sensing cuff 304, that is, pressure of the artery passage portion 90a of the left wrist 90, by the second pressure sensor 111, and acquires a pulse wave signal Pm as a fluctuation component. The blood pressure measurement unit 103F calculates a blood pressure value based on the pulse wave signal Pm by applying a known algorithm by an oscillometric method. After calculating the blood pressure value, the blood pressure measurement unit 103F stops the pump 113, opens the open/close valve 114, and performs control to discharge the fluid in the pressing cuff 302 and the sensing cuff 304.

The blood pressure measurement unit 103F can perform blood pressure measurement for each situation of the measurement subject by the above-described control. For example, the blood pressure measurement unit 103F can perform blood pressure measurement in the case where the estimation unit 103D estimates that the measurement subject is moving. For example, the blood pressure measurement unit 103F can perform blood pressure measurement in the case where the estimation unit 103D estimates that the measurement subject is staying at home. For example, the blood pressure measurement unit 103F can perform blood pressure measurement in the case where the estimation unit 103D estimates that the measurement subject is staying in the workplace. The blood pressure measurement unit 103F stores the blood pressure value in the memory 104 in association with the date and time of the blood pressure measurement and the situation of the measurement subject.

The blood pressure measurement unit 103F can acquire information on the date and time of the blood pressure measurement by the clock function of the blood pressure monitor 1. The blood pressure measurement unit 103F can acquire the situation of the measurement subject by referring to the estimation result by the estimation unit 103D.

The configuration of the designation information acquisition unit 103G will be described.

The designation information acquisition unit 103G acquires designation information including a designated place based on designation by the measurement subject and a past staying date and time range at the designated place. An example will be described. The measurement subject designates the designated place and the past staying date and time range at the designated place using the operation unit 102. The designated place is an estimation target of the staying place of the measurement subject by the blood pressure monitor 1. The staying date and time range is a range of date and time when the measurement subject stayed at the designated place in the past. For example, the measurement subject can designate a workplace as the designated place, and designate a specific staying start date and time and a specific end of staying date and time as a range of dates and times of staying in the workplace in the past.

The operation unit 102 outputs, to the CPU 103, the designation information including the designated place and the past staying date and time range at the designated place. Accordingly, the designation information acquisition unit 103G can acquire the designation information from the operation unit 102.

The designation information acquisition unit 103G outputs the designation information to the creation unit 103H.

The configuration of the creation unit 103H will be described.

The creation unit 103H creates an estimation condition used for estimating the stay at the designated place based on at least one of the activity amount or the number of steps in the time zone including the staying date and time range. Here, the activity amount will be taken as an example. The creation unit 103H can create the estimation condition based on the number of steps as in the example of the activity amount described here. Therefore, the description of the number of steps will be omitted.

The creation unit 103H acquires, from the memory 104, the activity amount data in the time zone including the staying date and time range included in the designation information. For example, the time zone including the staying date and time range is a time zone obtained by adding a predetermined time before and after the staying date and time range, but is not limited thereto. The creation unit 103H can acquire not only the activity amount during the stay at the designated place but also the activity amount in the process of arriving at the designated place and the process of leaving the designated place by using the activity amount in the time zone including the staying date and time range.

The creation unit 103H creates an estimation condition including at least one of a first change pattern of the activity amount in a process in which the measurement subject arrives at the designated place, a second change pattern of the activity amount during the stay of the measurement subject at the designated place, and a third change pattern of the activity amount in a process in which the measurement subject leaves the designated place, based on the activity amount in the time zone including the staying date and time range. For example, the first change pattern is a change (decrease) pattern of the activity amount per unit time in a predetermined time zone near the staying start date and time, but is not limited thereto. For example, the second change pattern is a change pattern of the activity amount per unit time in a predetermined time zone in the staying date and time range, but is not limited thereto. The predetermined time zone in the staying date and time range is a time zone in which the distribution of the activity amount per unit time changes characteristically. For example, the predetermined time zone in the staying date and time range is a time zone including a lunch break, but is not limited thereto. The third change pattern is a change (increase) pattern of the activity amount per unit time in a predetermined time zone near the end of staying date and time, but is not limited thereto.

The creation unit 103H outputs the estimation condition to the estimating unit 103D.

Examples of the life pattern candidates described above will now be described.

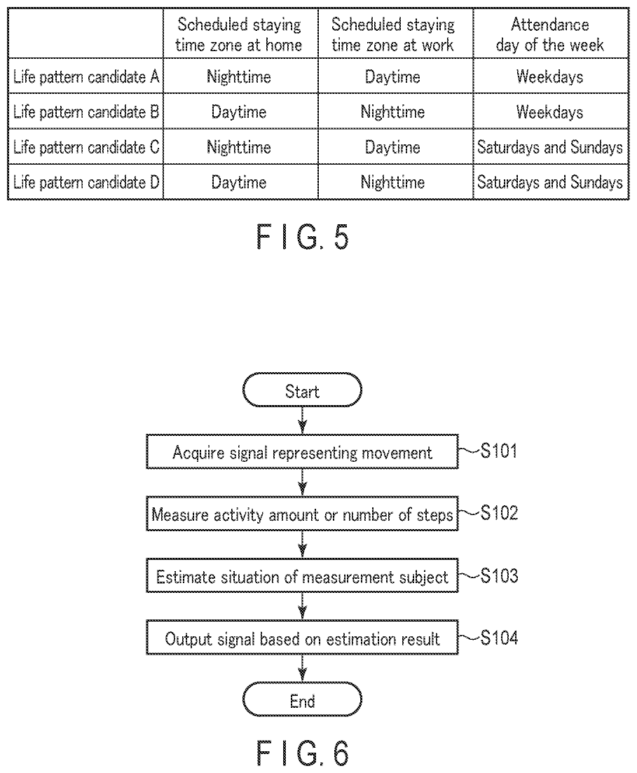

FIG. 5 is a diagram showing an example of a plurality of life pattern candidates. Note that the plurality of life pattern candidates shown here are examples, and the life pattern candidates are not limited thereto.

Each of the plurality of life pattern candidates shown in FIG. 5 is an example including a scheduled staying time zone at home, a scheduled staying time zone at work, and a day of the week of attendance. The life pattern candidates A, B, C, and D are different from each other. In the life pattern candidate A, the scheduled staying time zone at home is nighttime, the scheduled staying time zone at work is daytime, and the days of the week of attendance are weekdays. In the life pattern candidate B, the scheduled staying time zone at home is daytime, the scheduled staying time zone at work is nighttime, and the days of the week of attendance are weekdays. In the life pattern candidate C, the scheduled staying time zone at home is nighttime, the scheduled staying time zone at work is daytime, and the days of the week of attendance are Saturdays and Sundays.

In the life pattern candidate D, the scheduled staying time zone at home is daytime, the scheduled staying time zone at work is nighttime, and the days of the week of attendance are Saturdays and Sundays.

The measurement subject can display a plurality of life pattern candidates on the display unit 101 by operating the operation unit 102. The measurement subject can select a life pattern candidate close to his/her own life pattern from among the plurality of life pattern candidates. The CPU 103 stores the life pattern candidate selected by the measurement subject in the memory 104 as life pattern data of the measurement subject.

(Operation)

Estimation of the situation of the measurement subject performed by the blood pressure monitor 1 using at least one of the activity amount or the number of steps will be described.

FIG. 6 is a flowchart showing an example of a procedure for estimating the situation of the measurement subject and the contents thereof.

The signal acquisition unit 103A acquires a signal representing the movement of the measurement subject from a sensor that detects the movement of the measurement subject (step S101). In step S101, for example, the signal acquisition unit 103A acquires an acceleration signal from the acceleration sensor 105.

The measurement unit 103B measures at least one of the activity amount or the number of steps of the measurement subject based on the signal representing the movement of the measurement subject (step S102). In step S102, for example, the measurement unit 103B measures at least one of the activity amount or the number of steps of the measurement subject based on the acceleration signal.

The estimation unit 103D estimates the situation of the measurement subject based on at least one of the activity amount or the number of steps (step S103). The estimation of the situation of the measurement subject performed by the estimation unit 103D using at least one of the activity amount or the number of steps in step S103 will be described later.

The signal output unit 103E outputs a signal based on the estimation result by the estimation unit 103D (step S104). In step S104, for example, the signal output unit 103E outputs at least one of the instruction signal or the signal including the estimation result as the signal based on the estimation result. In the case where the signal output unit 103E outputs the instruction signal, the blood pressure measurement unit 103F can perform blood pressure measurement based on detection of the measurement switch being pressed by the measurement subject or detection of the instruction signal.

In the following, the above-described estimation of the situation of the measurement subject performed by the estimation unit 103D using at least one of the activity amount or the number of steps in step S103 will be described.

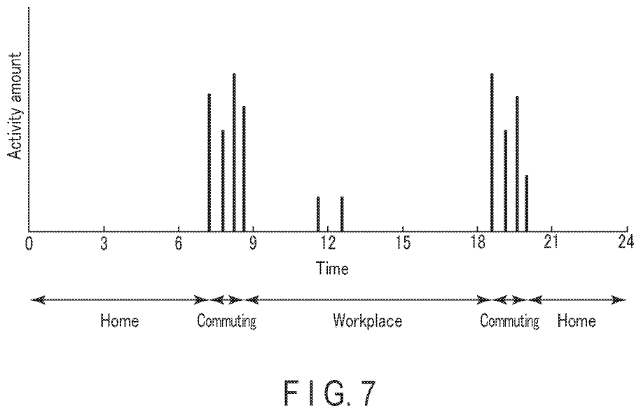

FIG. 7 is a diagram showing the distribution of the activity amount per unit time of a certain day of the measurement subject measured by the blood pressure monitor 1. The horizontal axis represents time. The vertical axis represents the amount of activity. In this example, the measurement subject moves for commuting from 7:00 to 9:00, stays at work from 9:00 to 18:00, moves for commuting from 18:00 to 20:00, and stays at home after 20:00.

In the case where the measurement subject walks or moves, the amount of activity per unit time is large. On the other hand, in the case where the measurement subject stays at some place and hardly moves, the amount of activity per unit time is small. For this reason, the amount of activity per unit time in the case where the measurement subject is staying at some place is smaller than the amount of activity per unit time in the case where the measurement subject is moving. In other words, the amount of activity per unit time varies depending on the situation of the measurement subject.

As described above, the activity amount data for one day has a characteristic that the activity amount per unit time varies depending on the situation of the measurement subject. The estimation unit 103D estimates the situation of the measurement subject based on the activity amount, for example, in the manner below.

In one example, the estimation unit 103D uses a reference value for estimating that the measurement subject is moving (hereinafter also referred to as a “movement estimation reference value”) and a reference value for estimating that the measurement subject is staying at some place (hereinafter also referred to as a “stay estimation reference value”). Each of the movement estimation reference value and the stay estimation reference value is, for example, an arbitrary fixed value, but may be a value that can be appropriately changed depending on the measurement subject. The stay estimation reference value may be the same as or smaller than the movement estimation reference value.

The estimation unit 103D uses the movement estimation reference value to estimate that the measurement subject is moving, for example, in the manner below. For example, in a case where the estimation unit 103D determines that the amount of activity per unit time is equal to or greater than the movement estimation reference value, the estimation unit 103D estimates that the measurement subject is moving. Alternatively, for example, in a case where the estimation unit 103D determines that the activity amount is equal to or greater than the movement estimation reference value in a plurality of consecutive unit times, the estimation unit 103D may estimate that the measurement subject is moving. This is because, even when the measurement subject is staying at some place, the activity amount may become equal to or greater than the movement estimation reference value in a unit time depending on the behavior of the measurement subject. Accordingly, the estimation unit 103D can reduce erroneous estimation of the situation of the measurement subject. For the same reason, the estimation unit 103D may estimate that the measurement subject is moving in the case where it is determined that the activity amount is equal to or greater than the movement estimation reference value in a predetermined number of unit times among a plurality of consecutive unit times.

The estimation unit 103D uses the stay estimation reference value to estimate that the measurement subject is staying at some place, for example, in the manner below. For example, in a case where the estimation unit 103D determines that the activity amount per unit time is smaller than the stay estimation reference value, the estimation unit 103D estimates that the measurement subject is staying at some place. Alternatively, for example, in a case where the estimation unit 103D determines that the activity amount is smaller than the stay estimation reference value in a plurality of consecutive unit times, it may be estimated that the measurement subject is staying at some place. This is because, even when the measurement subject is moving, the activity amount may be smaller than the stay estimation reference value in a unit time depending on the behavior of the measurement subject. Accordingly, the estimation unit 103D can reduce erroneous estimation of the situation of the measurement subject. For the same reason, the estimation unit 103D may estimate that the measurement subject is staying at some place in the case where it is determined that the activity amount is smaller than the stay estimation reference value in a predetermined number of unit times among a plurality of consecutive unit times.

In this manner, the estimation unit 103D can estimate that the measurement subject is moving and the measurement subject is staying as the situation of the measurement subject based on the variation in the activity amount per unit time.

In another example, the estimation unit 103D uses the amount of change in the activity amount of two consecutive unit times. For example, the estimation unit 103D detects the amount of change from the activity amount of the first unit time to the activity amount of the second unit time. The second unit time is a unit time continuous with the first unit time and is a unit time at a time later than the first unit time. The amount of change is, for example, a difference, but may also be a ratio.

The estimation unit 103D uses the amount of change in the activity amount of two consecutive unit times to estimate that the measurement subject is staying at some place, for example, in the manner below.

For example, in a case where the estimation unit 103D detects that a decrease in the amount of change in the activity amount is equal to or greater than the reference amount or the reference rate, the estimation unit 103D estimates that the situation of the measurement subject has transitioned from moving to staying. Each of the reference amount and the reference rate is, for example, an arbitrary fixed value, but may be a value that can be appropriately changed according to the measurement subject.

For example, after detecting that the decrease in the amount of change in the activity amount is equal to or greater than the reference amount or the reference rate, the estimation unit 103D monitors the amount of change in a plurality of continuously detected activity amounts. This is because, even in the case where the measurement subject is moving, the amount of change in the activity amount may temporarily decrease by an amount or rate equal to or greater than the reference amount or the reference rate depending on the behavior of the measurement subject. For example, in a case where the estimation unit 103D detects that the amount of change in a plurality of continuously detected activity amounts is smaller than the reference amount or the reference rate, the estimation unit 103D estimates that the situation of the measurement subject has transitioned from moving to staying. Alternatively, for example, the estimation unit 103D may estimate that the situation of the measurement subject has transitioned from moving to staying in a case where the amount of change in a predetermined number of activity amounts among the amount of change in a plurality of continuously detected activity amounts is determined to be smaller than the reference amount or the reference rate. Accordingly, the estimation unit 103D can reduce erroneous estimation of the situation of the measurement subject.

The estimation unit 103D uses the amount of change in the activity amount of two consecutive unit times to estimate that the measurement subject is moving, for example, in the manner below.

For example, in a case where the estimation unit 103D detects that an increase in the amount of change in the activity amount is equal to or greater than the reference amount or the reference rate, the estimation unit 103D estimates that the situation of the measurement subject has transitioned from staying to moving. Each of the reference amount and the reference rate is, for example, an arbitrary fixed value, but may be a value that can be appropriately changed according to the measurement subject.

For example, after detecting that the increase in the amount of change in the activity amount is equal to or greater than the reference amount or the reference rate, the estimation unit 103D monitors the amount of change in a plurality of continuously detected activity amounts. This is because, even in the case where the measurement subject is staying, the amount of change in the activity amount may temporarily increase by an amount or rate equal to or greater than the reference amount or the reference rate depending on the behavior of the measurement subject. For example, in a case where the estimation unit 103D detects that the amount of change in a plurality of continuously detected activity amounts is smaller than the reference amount or the reference rate, the estimation unit 103D estimates that the situation of the measurement subject has transitioned from staying to moving. Alternatively, for example, the estimation unit 103D may estimate that the situation of the measurement subject has transitioned from staying to moving in a case where the amount of change in a predetermined number of activity amounts among the amount of change in a plurality of continuously detected activity amounts is detected to be smaller than the reference amount or the reference rate. Accordingly, the estimation unit 103D can reduce erroneous estimation of the situation of the measurement subject.

In this manner, the estimation unit 103D can estimate that the measurement subject is moving and the measurement subject is staying as the situation of the measurement subject based on the variation in the activity amount per unit time.

Estimation of the staying place of the measurement subject by the estimation unit 103D will now be described.

In a case where the estimation unit 103D estimates that the measurement subject is staying at some place as described above, for example, it is possible to estimate the staying place of the measurement subject in the following manner. Note that the term “current date and time” in the following description may be read as “date and time at which the estimation unit 103D estimates that the measurement subject is staying” as appropriate. The estimation unit 103D can acquire information on the current date and time by a clock function of the blood pressure monitor 1. The estimation unit 103D can determine whether the current date and time is a weekday, a Saturday/Sunday, or a holiday (national holiday) with reference to the information on the current date and time and the calendar information stored in the memory 104.

In one example, the estimation unit 103D estimates the staying place of the measurement subject with reference to the life pattern data described above. Here, five different life pattern data will be described as an example.

(Example of First Life Pattern Data)

An example in which the life pattern data includes a scheduled staying time zone at home will be described.

In a case where the current date and time is included in the scheduled staying time zone at home, the estimation unit 103D estimates that the staying place of the measurement subject is at home. On the other hand, in a case where the current date and time is not included in the scheduled staying time zone at home, the estimation unit 103D estimates that the staying place of the measurement subject is a place different from home. Alternatively, the estimation unit 103D may determine whether or not the current date and time is included in a predetermined time before and after the scheduled staying time zone at home. This is because there is a possibility that the scheduled staying time zone included in the life pattern data may deviate from the actual staying time zone of the measurement subject. In a case where the current date and time is included in the predetermined time before and after the scheduled staying time zone at home, the estimation unit 103D estimates that the staying place of the measurement subject is at home. In a case where the current date and time is not included in the predetermined time before and after the scheduled staying time zone at home, the estimation unit 103D estimates that the staying place of the measurement subject is a place different from home.

(Example of Second Life Pattern Data)

An example in which the life pattern data includes a scheduled staying time zone in the workplace but does not include an attendance day of the week will be described.

In a case where the current date and time is included in the scheduled staying time zone in the workplace, the estimation unit 103D estimates that the staying place of the measurement subject is the workplace. Alternatively, in the case where the current date and time is included in the scheduled staying time zone in the workplace, the estimation unit 103D may determine whether the day of the week corresponding to the current date and time is a weekday. In the case where the day of the week corresponding to the current date and time is a weekday, the estimation unit 103D estimates that the staying place of the measurement subject is the workplace. This is because many people are likely to be at work on weekdays. On the other hand, in a case where the day of the week corresponding to the current date and time is not a weekday, the estimation unit 103D estimates that the staying place of the measurement subject is a place different from the workplace. This is because many people are unlikely to stay at work on days other than weekdays.

In a case where the current date and time is not included in the scheduled staying time zone in the workplace, the estimation unit 103D estimates that the staying place of the measurement subject is a place different from the workplace. Alternatively, as described above, the estimation unit 103D may estimate the staying place of the measurement subject in consideration of the relationship between the current date and time and the predetermined time before and after the scheduled staying time zone in the workplace and the day of the week corresponding to the current date and time.

(Example of Third Life Pattern Data)

An example in which the life pattern data includes a scheduled staying time zone and an attendance day of the week at work will be described.

In a case where the current date and time is included in the scheduled staying time zone in the workplace, the estimation unit 103D determines whether or not the day of the week corresponding to the current date and time is the attendance day of the week. In a case where the day of the week corresponding to the current date and time is the attendance day of the week, the estimation unit 103D estimates that the staying place of the measurement subject is the workplace. In a case where the day of the week corresponding to the current date and time is not the attendance day of the week, the estimation unit 103D estimates that the staying place of the measurement subject is a place different from the workplace. In a case where the current date and time is not included in the scheduled staying time zone in the workplace, the estimation unit 103D estimates that the staying place of the measurement subject is a place different from the workplace. Alternatively, as described above, the estimation unit 103D may estimate the staying place of the measurement subject in consideration of the relationship between the current date and time and the predetermined time before and after the scheduled staying time zone in the workplace and the relationship between the day of the week corresponding to the current date and time and the attendance day of the week.

(Example of Fourth Life Pattern Data)

An example in which the life pattern data includes a scheduled staying time zone at home, a scheduled staying time zone at work, and an attendance day of the week will be described. The life pattern data of this example corresponds to the life pattern candidates shown in FIG. 5.

In a case where the current date and time is included in the scheduled staying time zone at home, the estimation unit 103D estimates that the staying place of the measurement subject is at home. In a case where the current date and time is included in the scheduled staying time zone in the workplace, the estimation unit 103D estimates the staying place of the measurement subject in the manner described in the example of the third life pattern data. That is, the estimation unit 103D estimates that the staying place of the measurement subject is the workplace or a place different from the workplace in consideration of the relationship between the day of the week corresponding to the current date and time and the attendance day of the week.

In a case where the current date and time is included in neither of the scheduled staying time zone at home nor the scheduled staying time zone in the workplace, the estimation unit 103D performs, for example, the following processing.

In one example, the estimation unit 103D estimates that the staying place of the measurement subject is a place different from either the home or the workplace.

In another example, the estimation unit 103D determines whether the current date and time is close to the scheduled staying time zone at home or the scheduled staying time zone at work. In a case where the current date and time is closer to the scheduled staying time zone at home than the scheduled staying time zone at work, the estimation unit 103D estimates that the staying place of the measurement subject is at home. On the other hand, in a case where the current date and time is closer to the scheduled staying time zone at work than the scheduled staying time zone at home, the estimation unit 103D estimates the staying place of the measurement subject in consideration of the relationship between the day of the week corresponding to the current date and time and the attendance day of the week. That is, in a case where the day of the week corresponding to the current date and time is the attendance day of the week, the estimation unit 103D estimates that the staying place of the measurement subject is the workplace. On the other hand, in a case where the day of the week corresponding to the current date and time is not the attendance day of the week, the estimation unit 103D estimates that the staying place of the measurement subject is a place different from the workplace.

In yet another example, as described in the example of the first lifestyle pattern data, the estimation unit 103D estimates the staying place of the measurement subject in consideration of the relationship between the current date and time and the predetermined time before and after the scheduled staying time zone at home. Similarly, as described in the example of the third lifestyle pattern data, the estimation unit 103D estimates the staying place of the measurement subject in consideration of the relationship between the current date and time and the predetermined time before and after the scheduled staying time zone in the workplace and the relationship between the day of the week corresponding to the current date and time and the attendance day of the week.

(Example of Fifth Life Pattern Data)

An example in which the life pattern data includes a scheduled staying time zone at home and a scheduled staying time zone at work, but does not include the attendance day of the week will be described.

In a case where the current date and time is included in the scheduled staying time zone at home, the estimation unit 103D estimates that the staying place of the measurement subject is at home. In a case where the current date and time is included in the scheduled staying time zone in the workplace, the estimation unit 103D estimates the staying place of the measurement subject in the manner described in the example of the second life pattern data. That is, the estimation unit 103D estimates that the staying place of the measurement subject is the workplace or a place different from the workplace in consideration of the day of the week corresponding to the current date and time.

In a case where the current date and time is not included in either the scheduled staying time zone at home or the scheduled staying time zone at work, the estimation unit 103D performs, for example, the following processing.

In one example, the estimation unit 103D estimates that the staying place of the measurement subject is a place different from either the home or the workplace.

In another example, the estimation unit 103D determines whether the current date and time is close to the scheduled staying time zone at home or the scheduled staying time zone at work. In a case where the current date and time is closer to the scheduled staying time zone at home than the scheduled staying time zone at work, the estimation unit 103D estimates that the staying place of the measurement subject is at home. On the other hand, in a case where the current date and time is closer to the scheduled staying time zone at work than the scheduled staying time zone at home, the estimation unit 103D estimates the staying place of the measurement subject in consideration of the day of the week corresponding to the current date and time. That is, in a case where the day of the week corresponding to the current date and time is a weekday, the estimation unit 103D estimates that the staying place of the measurement subject is the workplace. On the other hand, in a case where the day of the week corresponding to the current date and time is a day other than a weekday, the estimation unit 103D estimates that the staying place of the measurement subject is a place different from the workplace.

In yet another example, the estimation unit 103D may estimate the staying place of the measurement subject in consideration of the relationship between the current date and time and the predetermined time before and after the scheduled staying time zone at home in the manner described in the example of the first life pattern data. Similarly, as described in the example of the second lifestyle pattern data, the estimation unit 103D estimates the staying place of the measurement subject in consideration of the relationship between the current date and time and the predetermined time before and after the scheduled staying time zone in the workplace and the day of the week corresponding to the current date and time.

An example in which the life pattern data includes the scheduled staying time zones related to three or more places is the same as the example of the fourth life pattern data and the example of the fifth life pattern data described above, and thus the description thereof will be omitted.

In this manner, the estimation unit 103D can accurately estimate the staying place of the measurement subject by referring to the life pattern data. In the case where the life pattern data includes the attendance day of the week, the estimation unit 103D can estimate the place of the measurement subject with higher accuracy. As the number of scheduled staying time zones included in the life pattern data increases, the estimation unit 103D can estimate the place of the measurement subject with higher accuracy.

If the life pattern data is set for each day of the week, the estimation unit 103D can refer to the life pattern data set for the day of the week corresponding to the current date and time. The measurement subject may spend each day of the week differently. For example, the measurement subject may work during the day on one day of the week and work at night on another day of the week. The estimation unit 103D can estimate the staying place of the measurement subject with higher accuracy by referring to the life pattern data set for each day of the week.

In another example, the estimation unit 103D estimates the staying place of the measurement subject without referring to the life pattern data, for example, in the following manner.

In one example, the estimation unit 103D estimates the staying place of the measurement subject with reference to the current date and time. In a case where the current date and time is included in the nighttime, the estimation unit 103D estimates that the staying place of the measurement subject is home. This is because many people are likely to stay at home at nighttime. In a case where the current date and time is included in the daytime on weekdays, the estimation unit 103D estimates that the staying place of the measurement subject is the workplace. This is because many people are likely to be at work in the daytime during weekdays. In a case where the current date and time is included in the daytime on weekdays, the estimation unit 103D may estimate the staying place of the measurement subject as a place different from home instead of estimating the staying place as the workplace. This is because a place where a person retired from work stays during the day of a weekday is not a workplace.

In another example, the estimation unit 103D estimates the staying place of the measurement subject with reference to the current date and time and the activity amount. In this example, the memory 104 stores in advance the total activity amount required for the measurement subject to move between a first place and a second place. The total activity amount is used to estimate whether the measurement subject has moved between the first place and the second place. For example, the memory 104 stores in advance a total activity amount (hereinafter, also referred to as “first total activity amount”) required for the measurement subject to move between the home and the workplace. The estimation unit 103D calculates a total activity amount (hereinafter, also referred to as “second total activity amount”) within a predetermined time after it is determined that the activity amount per unit time is equal to or greater than the movement estimation reference value mentioned above. For example, the predetermined time corresponds to a time required for the measurement subject to move between the home and the workplace, and is set in advance. The estimation unit 103D compares the second total activity amount with the first total activity amount. In a case where it is determined that the second total activity amount matches the first total activity amount or substantially matches the first total activity amount within a predetermined range, the estimation unit 103D estimates that the measurement subject has moved between the home and the workplace. In this case, the estimation unit 103D further estimates the staying place according to the current date and time, for example, in the following manner.

In a case where the current date and time is the morning of a weekday, the estimation unit 103D estimates that the measurement subject has moved from home to work. This is because many people are likely to go to work in the morning on weekdays. Accordingly, the estimation unit 103D can estimate the staying place of the measurement subject as the workplace after a time after which it is determined that the second total activity amount matches the first total activity amount or substantially matches the first total activity amount within the predetermined range. In a case where the current date and time is the afternoon of a weekday, the estimation unit 103D estimates that the measurement subject has moved from work to home. This is because many people are likely to return home in the afternoon on weekdays. Accordingly, the estimation unit 103D can estimate that the staying place of the measurement subject is at home after a time after which it is determined that the second total activity amount matches the first total activity amount or substantially matches the first total activity amount within the predetermined range.

In the following, the above-described estimation of the situation of the measurement subject performed by the estimation unit 103D with reference to the estimation condition in step S103 will be described.

The estimation unit 103D refers to the estimation condition and estimates that the measurement subject is staying at the designated place based on the activity amount. An example will be described. The estimation unit 103D compares the distribution of the activity amount per unit time with a plurality of change patterns included in the estimation condition. The estimation unit 103D determines whether the distribution of the activity amount per unit time matches or substantially matches any of the plurality of change patterns included in the estimation condition. For example, when the distribution of the activity amount per unit time is smaller than a predetermined rate of deviation from the change pattern, the estimation unit 103D can determine that the distribution of the activity amount substantially matches the change pattern.

In a case where the distribution of the activity amount per unit time matches or substantially matches the first change pattern included in the estimation condition, the estimation unit 103D estimates that the measurement subject is staying at the designated place. In a case where the distribution of the activity amount per unit time matches or substantially matches the second change pattern included in the estimation condition, the estimation unit 103D estimates that the measurement subject is staying at the designated place. In a case where the distribution of the activity amount per unit time matches or substantially matches the third change pattern included in the estimation condition, the estimation unit 103D estimates that the measurement subject is away from the designated place. That is, the estimation unit 103D estimates that the measurement subject is not staying at the designated place. On the other hand, in a case where the distribution of the activity amount per unit time does not match or does not substantially match any of the plurality of change patterns included in the estimation condition, the estimation unit 103D estimates that the measurement subject is not staying at the designated place.

The distribution of the number of steps per unit time is also similar to the distribution of the amount of activity per unit time shown in FIG. 7. Therefore, the estimation unit 103D can estimate the situation of the measurement subject based on the number of steps, similarly to the estimation of the situation of the measurement subject using the activity amount described above. For example, the estimation unit 103D can estimate that the measurement subject is moving and the measurement subject is staying as the situation of the measurement subject based on the variation in the number of steps per unit time.

The estimation unit 103D can also estimate the situation of the measurement subject based on both the activity amount and the number of steps. Accordingly, the estimation unit 103D can accurately estimate the situation of the measurement subject.

In this way, the estimation unit 103D can estimate the situation of the measurement subject based on at least one of the activity amount or the number of steps. For example, the estimation unit 103D can estimate that the measurement subject is moving and the measurement subject is staying as the situation of the measurement subject on the basis of a variation in at least one of the activity amount per unit time or the number of steps per unit time. For example, the estimation unit 103D can estimate that the measurement subject is staying at the designated place based on at least one of the activity amount or the number of steps with reference to the estimation condition.

(Effect)

As described above in detail, in one embodiment of the present invention, the blood pressure monitor 1 can estimate the situation of the measurement subject based on at least one of the activity amount or the number of steps of the measurement subject. As a result, the blood pressure monitor 1 can estimate the situation of the measurement subject with reference to the information from the already mounted sensor, and thus can estimate the situation of the measurement subject with a simple configuration. Furthermore, since the blood pressure monitor 1 does not need to refer to an external signal such as a GPS signal, it is possible to estimate the situation of the measurement subject even when the GPS signal cannot be acquired.

The blood pressure monitor 1 does not need to register the position information of various places for estimating the situation of the measurement subject in the memory 104 as in the case of estimating the situation of the measurement subject based on the GPS signal. Therefore, the blood pressure monitor 1 can effectively utilize the memory resources. Furthermore, for example, the blood pressure monitor 1 can acquire a blood pressure value in an estimated situation. As a result, the measurement subject can confirm their suspicion of high blood pressure in the estimated situation at an early stage.

Furthermore, in one embodiment of the present invention, the blood pressure monitor 1 can estimate that the measurement subject is moving and that the measurement subject is staying. Accordingly, the blood pressure monitor 1 can provide estimation results of different situations.

In addition, for example, the blood pressure monitor 1 can acquire a blood pressure value while the measurement subject is moving and a blood pressure value while the measurement subject is staying. As a result, the measurement subject can confirm their suspicion of high blood pressure at an early stage during movement (for example, while riding on a train). Similarly, the measurement subject can confirm their suspicion of high blood pressure at an early stage while staying at some place.

Furthermore, in one embodiment of the present invention, the blood pressure monitor 1 can estimate the staying place of the measurement subject with reference to the life pattern data. Accordingly, the blood pressure monitor 1 can estimate the staying place of the measurement subject with high accuracy. For example, the blood pressure monitor 1 can acquire a blood pressure value at each staying place of the measurement subject. As a result, the measurement subject can confirm their suspicion of high blood pressure at each staying place (for example, the workplace where high blood pressure is likely to occur) at an early stage.

Furthermore, in one embodiment of the present invention, the blood pressure monitor 1 can create an estimation condition based on at least one of the activity amount or the number of steps, and estimate that the measurement subject is staying at a designated place with reference to the estimation condition. Accordingly, the blood pressure monitor 1 can accurately estimate that the measurement subject is staying at the designated place by referring to the estimation condition based on at least one of the actually measured activity amount or the number of steps.

### Other Embodiments

As described above, the blood pressure monitor 1 is not limited to a blood pressure monitor of a type that starts blood pressure measurement based on an input of a blood pressure measurement start instruction by the measurement subject or a trigger signal autonomously generated by the blood pressure monitor 1. The blood pressure monitor 1 may be, for example, a blood pressure monitor employing a continuous measurement type blood pressure detection method using a pulse transmit time (PTT) method, a tonometry method, an optical method, a radio wave method, an ultrasonic method, or the like. The PTT method is a method of measuring a pulse transmit time (PTT) and estimating a blood pressure value from the measured pulse transmit time. The tonometry method is a method in which a pressure sensor is brought into direct contact with a biological site (measurement site) through which an artery such as a radial artery of a wrist passes, and a blood pressure value is measured using information detected by the pressure sensor. The optical method, the radio wave method, and the ultrasonic method are methods in which light, radio waves, or ultrasonic waves are applied to a blood vessel, and a blood pressure value is measured from reflected waves thereof.

The processing of the blood pressure monitor 1 described in one embodiment may be executed by an activity meter or a pedometer, which is an example of the information processing apparatus. That is, the CPU included in the activity meter or the pedometer may implement the signal acquisition unit 103A, the measurement unit 103B, the setting acquisition unit 103C, and the estimation unit 103D.

The processing of the blood pressure monitor 1 described in one embodiment may be executed by the external device 80, which is an example of the information processing apparatus. The CPU included in the external apparatus 80 may implement the signal acquisition unit 103A, the measurement unit 103B, the setting acquisition unit 103C, and the estimation unit 103D. In this case, the external device 80 can acquire an acceleration signal or the like from the blood pressure monitor 1 and execute the same processing as the processing of each unit implemented by the CPU 103 described above.

In short, the present invention is not limited to the exact embodiments described above, and can be embodied by modifying the constituent elements without departing from the gist of the invention at the implementation stage. Furthermore, various inventions can be formed by appropriately combining a plurality of constituent elements disclosed in the above embodiments. For example, some of the constituent elements may be deleted from the entire set of constituent elements shown in the embodiment. Moreover, constituent elements described in different embodiments may be suitably combined.

The various functional units described in the above embodiments may be realized by using circuits. The circuit may be a dedicated circuit that realizes a specific function or may be a general-purpose circuit such as a processor.

At least a part of the processing of each of the above embodiments can also be realized by using a general-purpose computer as basic hardware. The program for realizing the above-described processing may be provided by being stored in a computer-readable recording medium. The program is stored in the recording medium as a file in an installable format or a file in an executable format. Examples of the recording medium include a magnetic disk, an optical disc (such as a compact disc-read only memory (CD-ROM), a compact disc-recordable (CD-R), and a digital versatile disc (DVD)), a magneto-optical disc (such as a magneto optical (MO)), and a semiconductor memory. The recording medium may be any medium as long as it can store the program and can be read by a computer. Furthermore, the program for realizing the above-described processing may be stored in a computer (server) connected to a network such as the Internet and downloaded to a computer (client) via the network.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Some or all of the above embodiments may be described as in the following supplementary notes, but are not limited thereto.

(Supplementary Note 1)

An information processing apparatus comprising:

a processor configured to

acquire a signal representing a movement of a subject from a sensor that detects the movement of the subject,

measure at least one of an activity amount or a number of steps of the subject based on the signal representing the movement of the subject, and

estimate a situation of the subject based on at least one of the activity amount or the number of steps; and

a memory that stores a command to cause the processor to operate.

(Supplementary Note 2)

An information processing method comprising:

acquiring, by using at least one processor, a signal representing a movement of a subject from a sensor detecting the movement of the subject;

measuring, by using the at least one processor, at least one of an activity amount or a number of steps of the subject based on the signal representing the movement of the subject; and

estimating, by using the at least one processor, a situation of the subject based on the at least one of the activity amount or the number of steps.

(Supplementary Note 3)

An information processing apparatus comprising:

a signal acquisition unit (103A) configured to acquire a signal representing a movement of a subject from a sensor configured to detect the movement of the subject;

a measurement unit (103B) configured to measure at least one of an activity amount or a number of steps of the subject based on the signal representing the movement of the subject; and

an estimation unit (103D) configured to estimate a situation of the subject based on at least one of the activity amount or the number of steps.

### REFERENCE SIGNS LIST

1 . . . blood pressure monitor
10 . . . main body
10A . . . case
10B . . . glass
10C . . . back cover
20 . . . belt
30 . . . cuff structure
30a . . . one end
30b . . . the other end
30c . . . inner peripheral surface
80 . . . external device
90 . . . left wrist
91 . . . radial artery
92 . . . ulnar artery
93 . . . radius
94 . . . ulna
95 . . . tendon
101 . . . display unit
102 . . . operation unit
103 . . . CPU
103A . . . signal acquisition unit
103B . . . measurement unit
103C . . . setting acquisition unit
103D . . . estimation unit
103E . . . signal output unit
103F . . . blood pressure measurement unit
103G . . . designation information acquisition unit
103H . . . creation unit
104 . . . memory
105 . . . acceleration sensor
106 . . . temperature and humidity sensor
107 . . . atmospheric pressure sensor
108 . . . communication unit
109 . . . battery
110 . . . first pressure sensor
111 . . . second pressure sensor
112 . . . pump drive circuit
113 . . . pump
114 . . . open/close valve
201 . . . first belt portion
201a . . . root portion
201b . . . distal end portion
202 . . . second belt portion
202a . . . root portion
202b . . . distal end portion
202c . . . small hole
203 . . . buckle
203A . . . frame-shaped body
203B . . . fastening rod
203C . . . connecting rod
204 . . . belt holding portion
301 . . . curler
302 . . . pressing cuff
303 . . . back plate
304 . . . sensing cuff
304A . . . first sheet
304B . . . second sheet
401 . . . connecting rod
402 . . . connecting rod
501 . . . flexible tube
502 . . . flexible tube
503 . . . first flow path forming member
504 . . . second flow path forming member

1. An apparatus comprising processing circuitry coupled to a memory, the processing circuitry configured to:
acquire a signal representing a movement of a subject from a sensor that detects the movement of the subject;
measure at least one of an activity amount or a number of steps of the subject based on the signal representing the movement of the subject; and
estimate a situation of the subject staying or moving with respect to at least one place based on a pattern of variation and a current date and time of at least one of the activity amount or the number of steps.

2. The apparatus according to claim 1, wherein the processing circuitry is further configured to estimate, as the situation of the subject staying or moving with respect to the at least one place, that the subject is moving to the at least one place and that the subject is staying at the at least one place, based on the pattern of variation and the current date and time of at least one of the activity amount or the number of steps per unit time.

3. The apparatus according to claim 1, wherein the processing circuitry is further configured to:
acquire life pattern data including a scheduled staying time zone related to at least one place of the subject, and
estimate a staying place of the subject with reference to the life pattern data in a case where it is estimated that the subject is staying.

4. The apparatus according to claim 1, wherein the processing circuitry is further configured to:
acquire designation information including a designated place based on designation by the subject and a past staying date and time range at the designated place; and
create an estimation condition used for estimating that the subject is staying at the designated place based on at least one of the activity amount or the number of steps in a time zone including the staying date and time range, and
estimate that the subject is staying at the designated place by referring to the estimation condition.

5. A method comprising:
acquiring a signal representing a movement of a subject from a sensor that detects the movement of the subject;
measuring at least one of an activity amount or a number of steps of the subject based on the signal representing the movement of the subject; and
estimating a situation of the subject staying or moving with respect to at least one place based on a pattern of variation and a current date and time of at least one of the activity amount or the number of steps.

6. A non-transitory computer readable medium storing a computer program which is executed by a computer to provide the steps of:
acquiring a signal representing a movement of a subject from a sensor that detects the movement of the subject;
measuring at least one of an activity amount or a number of steps of the subject based on the signal representing the movement of the subject; and
estimating a situation of the subject staying or moving with respect to at least one place based on a pattern of variation and a current date and time of at least one of the activity amount or the number of steps.
